(19) United States
(12) Patent Application Publication
(10) Pub. No.: US 2024/0041796 A1
(43) Pub. Date: Feb. 8, 2024

(54) ANTI-INFLAMMATORY COMPOSITION COMPRISING N-BENZYL-N-METHYLDECAN-1-AMINE OR A DERIVATIVE THEREOF AS AN ACTIVE INGREDIENT

(71) Applicants: PUSAN NATIONAL UNIVERSITY INDUSTRY-UNIVERSITY COOPERATION FOUNDATION, Busan (KR); HaYoung Meditech Inc., Busan (KR)

(72) Inventor: Young Hwa CHUNG, Busan (KR)

(73) Assignees: PUSAN NATIONAL UNIVERSITY INDUSTRY-UNIVERSITY COOPERATION FOUNDATION, Busan (KR); HaYoung Meditech Inc., Busan (KR)

(21) Appl. No.: 18/363,123

(22) Filed: Aug. 1, 2023

(30) Foreign Application Priority Data
Aug. 1, 2022 (KR) 10-2022-0095425
May 9, 2023 (KR) 10-2023-0059573

(51) Int. Cl. A61K 31/137; A61P 29/00; A61P 1/00; A61P 19/02; A61P 17/00; A61K 8/41; A61Q 19/00

(52) U.S. Cl. CPC A61K 31/137; A61P 29/00; A61P 1/00; A61P 19/02; A61P 17/00; A61K 8/41; A61Q 19/007

(57) ABSTRACT

Disclosed is an anti-inflammatory composition containing N-benzyl-N-methyldecan-1-amine or a derivative thereof as an active ingredient. The anti-inflammatory composition contains a compound represented by a following Chemical Formula 1 as an active ingredient.

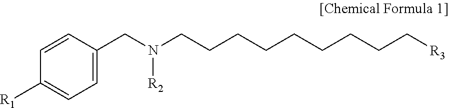

wherein in the Chemical Formula 1, R₁ represents hydrogen, fluorine, an alkyl group having 1 to 5 carbon atoms, or an alkoxy group having 1 to 5 carbon atoms, each of R₂ and R₃ independently represents an alkyl group having 1 to 5 carbon atoms.

### CROSS-REFERENCE TO RELATED APPLICATIONS

This application claims priority to Korean Patent Application No. 10-2022-0095425 filed on Aug. 1, 2022 in the Korean Intellectual Property Office and Korean Patent Application No. 10-2023-0059573 filed on May 9, 2023, and all the benefits accruing therefrom under 35 U.S.C. 119, the contents of each of which are herein incorporated by reference in their entirety.

### BACKGROUND

### Field

The present disclosure relates to an anti-inflammatory composition containing N-benzyl-N-methyldecan-1-amine or a derivative thereof as an active ingredient.

### Description of Related Art

Inflammation refers to the pathological condition of an abscess formed due to the invasion of external bacteria. Inflammatory response refers to a biological defense response process for repairing and regenerating damage caused by invasion that causes any organic change in cells or tissues of the living body. In case of tissue (cell) damage or infection by external infectious agents (bacteria, fungi, viruses, various types of allergens), various inflammatory mediators and immune cells in local blood vessels and body fluids are involved, resulting in a series of complex physiological reactions such as enzyme activation, inflammatory mediator secretion, body fluid infiltration, cell migration, and tissue destruction, and external symptoms such as erythema, edema, fever, and pain.

In normal cases, the inflammatory response removes external infectious agents and regenerates damaged tissues to restore life functions. However, when the antigen is not removed or the internal substance causes an excessive or continuous inflammatory response, this results in a major pathological phenomenon of the disease (hypersensitivity disease, chronic inflammation), and becomes an obstacle in the treatment process such as blood transfusion, drug administration, and organ transplantation.

In order to alleviate these inflammatory symptoms, non-steroidal drugs such as ibuprofen and indomethacin are used, and steroidal drugs such as dexamethasone are used. However, side effects thereof in terms of safety have been reported, and thus, use thereof is limited.

Therefore, it is necessary to develop a safe anti-inflammatory substance with maximum effect and minimum side effects.

### SUMMARY

One purpose of the present disclosure is to provide an anti-inflammatory composition containing N-benzyl-N-methyldecan-1-amine or a derivative thereof as an active ingredient.

One purpose of the present disclosure is to provide a cosmetic or pharmaceutical composition containing N-benzyl-N-methyldecan-1-amine or a derivative thereof as an active ingredient for ameliorating atopic dermatitis.

One purpose of the present disclosure is to provide a pharmaceutical composition for preventing or treating rheumatoid arthritis, which contains N-benzyl-N-methyldecan-1-amine or a derivative thereof as an active ingredient.

One purpose of the present disclosure is to provide a medicinal composition for treating ulcerative colitis, which contains N-benzyl-N-methyldecan-1-amine or a derivative thereof as an active ingredient.

Purposes of the present disclosure are not limited to the above-mentioned purpose. Other purposes and advantages of the present disclosure that are not mentioned may be understood based on following descriptions, and may be more clearly understood based on embodiments of the present disclosure. Further, it will be easily understood that the purposes and advantages of the present disclosure may be realized using means shown in the claims and combinations thereof.

An anti-inflammatory composition according to one aspect of the present disclosure contains a compound represented by a following Chemical Formula 1 as an active ingredient:

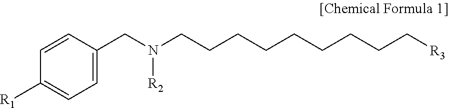

wherein in the Chemical Formula 1,
R₁ represents hydrogen, fluorine, an alkyl group having 1 to 5 carbon atoms, or an alkoxy group having 1 to 5 carbon atoms,
each of R₂ and R₃ independently represents an alkyl group having 1 to 5 carbon atoms.

In one embodiment, the anti-inflammatory composition contains one selected from compounds respectively represented by following Chemical Formulas 1-1 to 1-5 as the active ingredient:

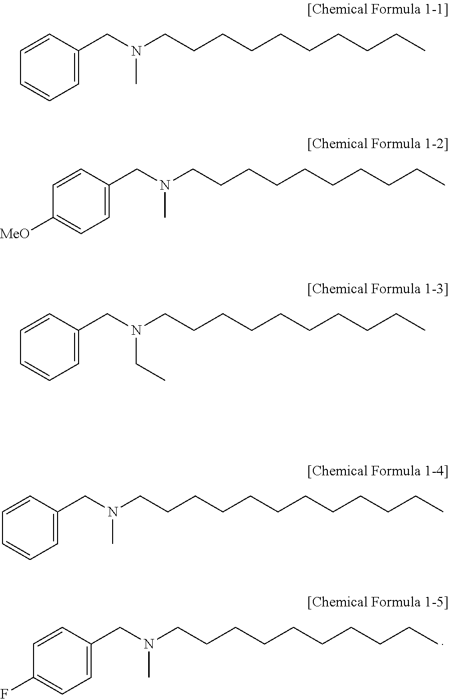 [SMILES: CCCCCCCCCCN(C)Cc1ccccc1 ; CCCCCCCCCCN(C)Cc1ccc(OC)cc1 ; CCCCCCCCCCN(CC)Cc1ccccc1 ; CCCCCCCCCCCCN(C)Cc1ccccc1 ; CCCCCCCCCCN(C)Cc1ccc(F)cc1]

In one embodiment, the anti-inflammatory composition has a therapeutic effect on an inflammatory disease including atopic dermatitis, rheumatoid arthritis, or ulcerative colitis.

In one embodiment, the anti-inflammatory composition inhibits activation of p38 MAP kinase and JNK induced by lipopolysaccharide, and inhibits an inflammation-related signal transduction pathway linked to p38 MAPK-MK2.

In one embodiment, the anti-inflammatory composition inhibits production of pro-inflammatory cytokines such as TNF-α, IL-1β, and IL-6, and inhibits activity of a transcription factor NF-_{K}B.

In one embodiment, the anti-inflammatory composition increases expression of HO-1 (Heme oxygenase 1) or Nrf2 (nuclear factor erythroid-related factor 2), thereby achieving an antioxidant effect.

In one embodiment, the anti-inflammatory composition inhibits production of chemokine CINC-3.

In one embodiment, the composition may be formulated into a variety of oral or parenteral dosage forms. When the composition is formulated, the formation may be prepared using diluents or excipients such as fillers, extenders, binders, wetting agents, disintegrants, and surfactants as commonly used. Solid preparations for oral administration may include tablets, pills, powders, granules, capsules, etc. Such solid preparations may be prepared by mixing at least one excipient, for example, starch, calcium carbonate, sucrose, lactose, gelatin, or the like with at least one compound. In addition to simple excipients, lubricants such as magnesium stearate and talc may also be used. Liquid preparations for oral administration may include suspensions, internally taking solutions, emulsions, syrups, etc. In addition to water and liquid paraffin as commonly used simple diluents, various excipients such as wetting agents, sweeteners, aromatics, and preservatives may be contained therein.

Formulations for parenteral administration may include sterilized aqueous solutions, non-aqueous solvents, suspensions, emulsions, freeze-dried formulations, and suppositories. Propylene glycol, polyethylene glycol, vegetable oils such as soybean oil and olive oil, and injectable esters such as ethyl oleate may be used as non-aqueous solvents and suspending agents. Witepsol, macrogol, tween 61, cacao butter, laurin paper, glycerogelatin, and the like may be used as a base for the suppository.

The composition according to the present disclosure may be administered to mammals such as rats, mice, livestock, and humans through various routes, for example, parenterally or orally. All modes of administration may be considered. For example, oral or rectal administration or intravenous, intramuscular, subcutaneous, intrauterine intrathecal or intracerebroventricular injection and the like may be used.

Further, another aspect of the present disclosure may provide a cosmetic composition for ameliorating atopic dermatitis, the composition containing a compound represented by a following Chemical Formula 2 as an active ingredient:

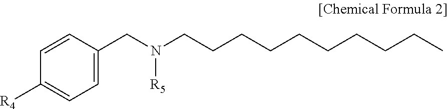

wherein in the Chemical Formula 2,
R₄ represents hydrogen, fluorine, an alkyl group having 1 to 5 carbon atoms, or an alkoxy group having 1 to 5 carbon atoms,
R₅ represents an alkyl group having 1 to 5 carbon atoms.

In one embodiment, the cosmetic composition contains one selected from compounds respectively represented by following Chemical Formulas 1-1, 1-2, 1-3 and 1-5 as the active ingredient:

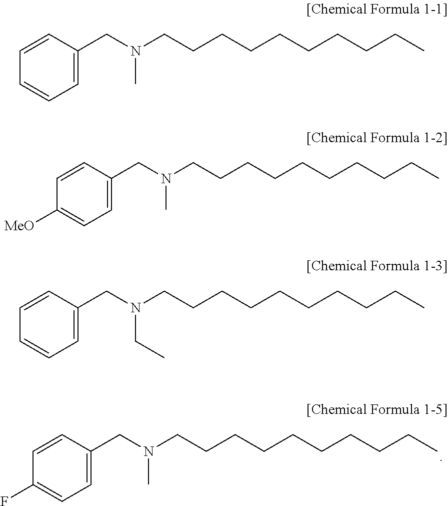 [SMILES: CCCCCCCCCCN(C)Cc1ccccc1 ; CCCCCCCCCCN(C)Cc1ccc(OC)cc1 ; CCCCCCCCCCN(CC)Cc1ccccc1 ; CCCCCCCCCCN(C)Cc1ccc(F)cc1]

In one embodiment, the cosmetic composition further contains at least one selected from a group consisting of a skin moisturizing ingredient, a cosmetic formulation ingredient, a fragrance, a preservative, and a purified water.

In one embodiment, a formulation of the cosmetic composition is lotion, cream, emulsion, essence, gel, serum, pack, powder, skin ointment, skin patch, suspension, spray, or cosmetic solution.

Further, still another aspect of the present disclosure may provide a pharmaceutical composition for preventing or treating rheumatoid arthritis, the composition containing, as an active ingredient, a compound represented by a following Chemical Formula 1 or a pharmaceutically acceptable salt thereof:

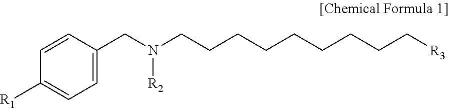

wherein in the Chemical Formula 1,
R₁ represents hydrogen, fluorine, an alkyl group having 1 to 5 carbon atoms, or an alkoxy group having 1 to 5 carbon atoms,
each of R₂ and R₃ independently represents an alkyl group having 1 to 5 carbon atoms.

In one embodiment, the pharmaceutical composition contains a compound represented by a following Chemical Formula 1-1 or Chemical Formula 1-2 as the active ingredient:

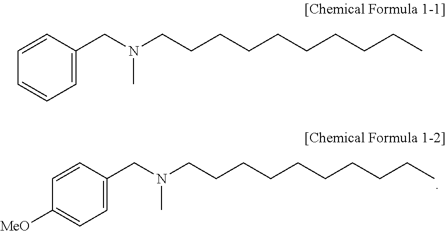 [SMILES: CCCCCCCCCCN(C)Cc1ccccc1 ; CCCCCCCCCCN(C)Cc1ccc(OC)cc1]

In one embodiment, the compound represented by Chemical Formula 1 is the compound represented by the Chemical Formula 1-1.

In one embodiment, the pharmaceutical composition is administered orally. In one embodiment, the compound represented by the Chemical Formula 1 is formulated in a unit dosage form suitable for oral administration at a dose of 15 to 25 mg/kg (in an amount of about 500 or 200 μl volume per dose).

In one embodiment, the pharmaceutical composition may be administered orally in a divided manner at one to several times. A dosage to a specific patient may vary depending on the patient's weight, age, sex, health condition, diet, administration time, administration method, excretion rate, and severity of the disease. etc.

In one embodiment, oral administration of the pharmaceutical composition for 3 weeks may have no effect on the activities of AST and ALT enzymes in a type 2 collagen-induced rheumatoid autoimmune disease model.

Further, still another aspect of the present disclosure may provide a medicinal composition for treatment of ulcerative colitis, the composition containing, as an active ingredient, a compound represented by a following Chemical Formula 1 or a pharmaceutically acceptable salt thereof:

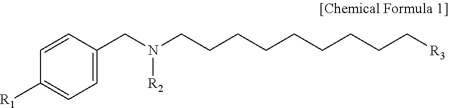

wherein in the Chemical Formula 1,
R₁ represents hydrogen, fluorine, an alkyl group having 1 to 5 carbon atoms, or an alkoxy group having 1 to 5 carbon atoms,
each of R₂ and R₃ independently represents an alkyl group having 1 to 5 carbon atoms.

In one embodiment, the medicinal composition contains, as the active ingredient, a compound represented by a following Chemical Formula 1-1 or Chemical Formula 1-2:

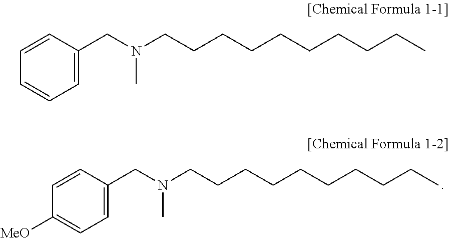 [SMILES: CCCCCCCCCCN(C)Cc1ccccc1 ; CCCCCCCCCCN(C)Cc1ccc(OC)cc1]

In one embodiment, the compound represented by the Chemical Formula 1 is the compound represented by the Chemical Formula 1-2.

In one embodiment, the medicinal composition is administered in an enema manner. In one embodiment, the medicinal composition is administered directly to an ulcerative colitis patient in the enema manner at 0.4 mg/kg or smaller (in an amount of about 100 μg or 500 μL volume per dose) as an effective dosage per day.

In one embodiment, the medicinal composition may be administered in an enema manner in a divided manner at one to several times. A dosage to a specific patient may vary depending on the patient's weight, age, sex, health condition, diet, administration time, administration method, excretion rate, and severity of the disease, etc.

In one embodiment, a dosage form of the medicinal composition may include the form of the pharmaceutically acceptable salt thereof. The medicinal composition may also be used alone or in combination with other pharmacologically active compounds, or in a suitable combination. The pharmaceutically acceptable salt is not particularly limited as long as the salt is pharmaceutically acceptable. For example, hydrochloric acid, sulfuric acid, nitric acid and the like may be used as the salt.

In one embodiment, the administration of the medicinal composition in an enema manner may suppress production of chemokine CINC-3. Further, the administration of the medicinal composition in an enema manner may reduce production of reactive oxygen species in macrophages.

According to the present disclosure, the N-benzyl-N-methyldecan-1-amine or the derivative thereof inhibits the inflammatory response of cells caused by lipopolysaccharide (LPS), and reduces the production of reactive oxygen species in macrophages, and inhibits the expression of chemokine CNIC-3, pro-inflammatory cytokines, and inflammatory complex proteins, thereby achieving an excellent anti-inflammatory effect. In particular, the N-benzyl-N-methyldecan-1-amine or the derivative thereof has an anti-inflammatory effect on inflammatory diseases such as atopic dermatitis, rheumatoid arthritis, and ulcerative colitis.

Specifically, the N-benzyl-N-methyldecan-1-amine or the derivative thereof according to the present disclosure may be applied to the skin to obtain an excellent anti-inflammatory effect against atopic dermatitis induced by 1,4-dinitrochlorobenzne (DNCB) compound. In addition, the N-benzyl-N-methyldecan-1-amine or the derivative thereof according to the present disclosure may be administered orally and thus exhibit excellent anti-inflammatory effect on rheumatoid arthritis induced from type 2 collagen. Moreover, the N-benzyl-N-methyldecan-1-amine or the derivative thereof according to the present disclosure may be administered in the enema manner and thus exhibit excellent anti-inflammatory effect on ulcerative colitis.

Effects of the present disclosure are not limited to the above-mentioned effects, and other effects as not mentioned will be clearly understood by those skilled in the art from following descriptions.

### BRIEF DESCRIPTION OF DRAWINGS

FIG. 1A shows a synthesis result and a structure based on H1-NMR of each of N-benzyl-N-methyldecan-1-amine (BMDA) and FIG. 1B shows a derivative thereof (DMMA) synthesized according to one embodiment of the present disclosure.

FIGS. 2A-D show an inhibitory effect of N-benzyl-N-methyldecan-1-amine (BMDA) and a derivative thereof (DMMA) according to one embodiment of the present disclosure inhibiting the inflammatory response of the monocyte cell line THP-1 by lipopolysaccharide (LPS).

FIGS. 3A-E show the anti-inflammatory effect via administration of N-benzyl-N-methyldecan-1-amine (BMDA) and a derivative thereof (DMMA) according to one embodiment of the present disclosure into the colitis model induced by DNBS in an enema manner.

FIGS. 4A-D and 5A-B show the experimental results showing the inflammatory effect via oral administration of N-benzyl-N-methyldecan-1-amine (BMDA) and a derivative thereof (DMMA) according to one embodiment of the present disclosure into a type 2 collagen-induced rheumatoid arthritis model.

FIGS. 6A-B show an examination result based on qRT-PCR and immunoblotting of each off expression of an inflammatory cytokine and expression of each of HO-1 (Heme oxygenase-1) and Nrf2 (nuclear factor erythroid-related factor 2) as antioxidant genes via oral administration of N-benzyl-N-methyldecan-1-amine (BMDA) and a derivative thereof (DMMA) according to one embodiment of the present disclosure into a type 2 collagen-induced rheumatoid arthritis model.

FIG. 7 shows AST and ALT activity measurement results via oral administration of N-benzyl-N-methyldecan-1-amine (BMDA) and a derivative thereof (DMMA) according to one embodiment of the present disclosure into a type 2 collagen-induced rheumatoid arthritis model.

FIGS. 8A-B show an inhibitory effect of each of derivatives of N-benzyl-N-methyldecan-1-amine (BMDA) (Benzyl-decyl-methyl-amine, Benzyl-dodecyl-methyl-amine, decyl-(4-fluoro-benzyl)-methyl-amine) according to one embodiment of the present disclosure inhibiting the inflammatory response of the monocyte cell line THP-1 by lipopolysaccharide (LPS).

FIGS. 9A-D show an evaluation result of the atopic dermatitis ameliorating efficacy of a cosmetic composition containing each of N-benzyl-N-methyldecan-1-amine (BMDA) and a derivative thereof according to one embodiment of the present disclosure.

FIGS. 10A-D and 11A-D are detailed results that further support the results of FIGS. 9A and 9B.

### DETAILED DESCRIPTIONS

Advantages and features of the present disclosure, and a method of achieving the advantages and features will become apparent with reference to embodiments described later in detail together with the accompanying drawings. However, the present disclosure is not limited to the embodiments as disclosed below, but may be implemented in various different forms. Thus, these embodiments are set forth only to make the present disclosure complete, and to completely inform the scope of the present disclosure to those of ordinary skill in the technical field to which the present disclosure belongs, and the present disclosure is only defined by the scope of the claims.

For simplicity and clarity of illustration, elements in the drawings are not necessarily drawn to scale. The same reference numbers in different drawings represent the same or similar elements, and as such perform similar functionality. Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure. Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

A shape, a size, a ratio, an angle, a number, etc. disclosed in the drawings for describing embodiments of the present disclosure are illustrative, and the present disclosure is not limited thereto. The same reference numerals refer to the same elements herein. Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “including”, “include”, and “including” when used in this specification, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of associated listed items. Expression such as “at least one of” when preceding a list of elements may modify the entire list of elements and may not modify the individual elements of the list. In interpretation of numerical values, an error or tolerance therein may occur even when there is no explicit description thereof.

In interpreting a numerical value, the value is interpreted as including an error range unless there is no separate explicit description thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, various examples and experimental examples of the present disclosure will be described in detail. However, the following examples are merely some examples of the present disclosure, and the present disclosure should not be construed as being limited to the following examples.

### Example 1: Structure and Synthesis Identification of BMDA (FMJ-G0) and a Derivative Thereof (FMJ-G4)

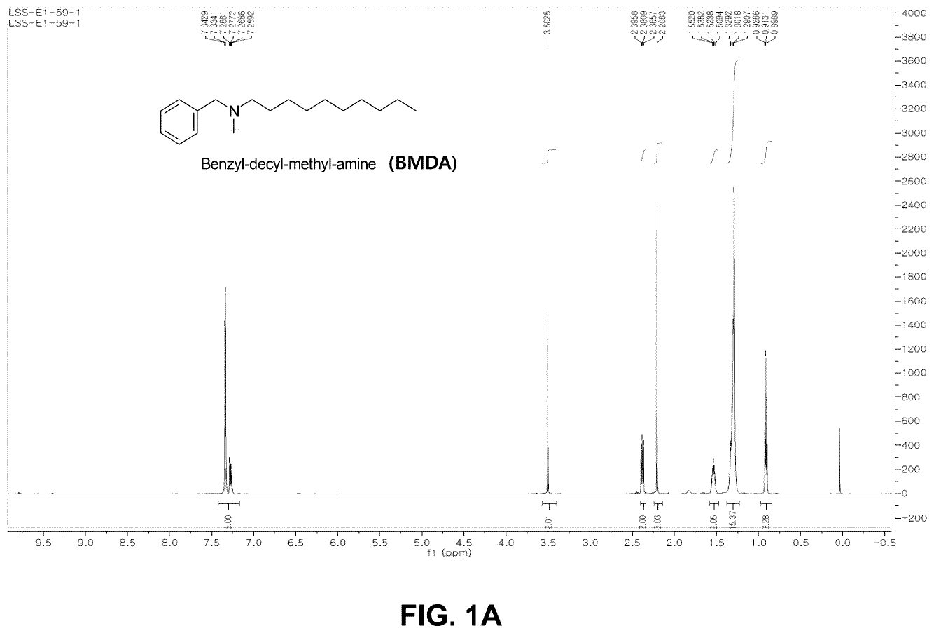

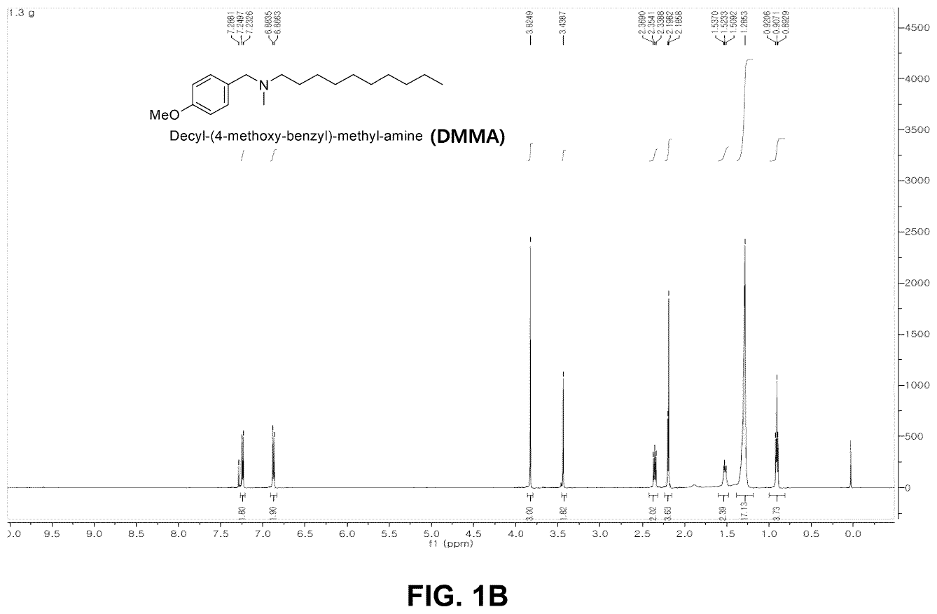

1M of N-benzylmethylamine and decaldehyde (decanal) were mixed with each other in presence of methanol and the mixture was stirred for 20 minutes. Thereafter, sodium triacetoxyborohydride [NaBH(OAc)₃] was added thereto and the mixture was further stirred for 1 hour. A volatile organic solvent was removed therefrom under vacuum and impure BMDA (Benzyl-decyl-methyl-amine) was extracted with ethyl acetic acid saturated with sodium hydrogen carbonate. More pure BMDA was obtained via purification using column chromatography, and a structure thereof was identified based on H′-NMR (see FIG. 1A). The chemical structure of the synthesized substance is represented by a following Chemical Formula 1-1.

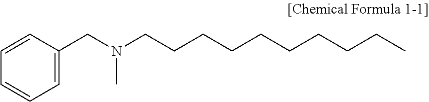 [SMILES: CCCCCCCCCCN(C)Cc1ccccc1]

In another example, the same process was performed using 1-(4-methoxyphenyl)-N-methylmethanamine and decaldehyde (decanal) as starting materials. Thus, FMJ-G4 substance (DMMA, Decyl-(4-methoxy-benzyl)-methyl-amine) was synthesized, and a structure thereof was also identified based on H′-NMR (see FIG. 1B). The chemical structure of the synthesized substance is represented by a following Chemical Formula 1-2.

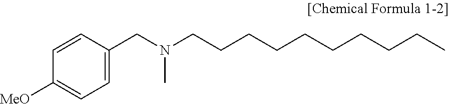 [SMILES: CCCCCCCCCCN(C)Cc1ccc(OC)cc1]

### Example 2: Evaluation of Anti-Inflammatory Efficacy of Each of BMDA (FMJ-G0) and a Derivative Thereof (DMMA, FMJ-G4)

1. Evaluation of efficacy of BMDA (FMJ-G0) and the derivative (DMMA) thereof in inhibiting the activation of the inflammation-related signal transduction induced by lipopolysaccharide (LPS) and inhibiting the production of TNF-α and IL-β inflammatory cytokines

Each of BMDA (FMJ-G0) or DMMA (FMJ-G4) was added to a culture medium at concentrations of 41.1M, 21.1M, and 1 μM in a 6-well plate. The human THP-1 monocytic cell line (5×10⁵ cells/well) was cultured therein for 3 hours. Each well was treated with lipopolysaccharide at a concentration of 1 μg/ml. After 1 hour, centrifugation was performed to obtain a supernatant which was used in an ELISA for inflammatory cytokine evaluation. Cell lysate was added to the collected THP-1 cells for immunoblotting.

Moreover, the mononuclear THP-1 cell line treated with lipopolysaccharide (LPS) and BMDA/DMMA under the same conditions was partitioned into cytoplasmic and nuclear portions. Distribution of NF-_{K}B P65 protein in the cytoplasm and nucleus was examined based on immunoblotting.

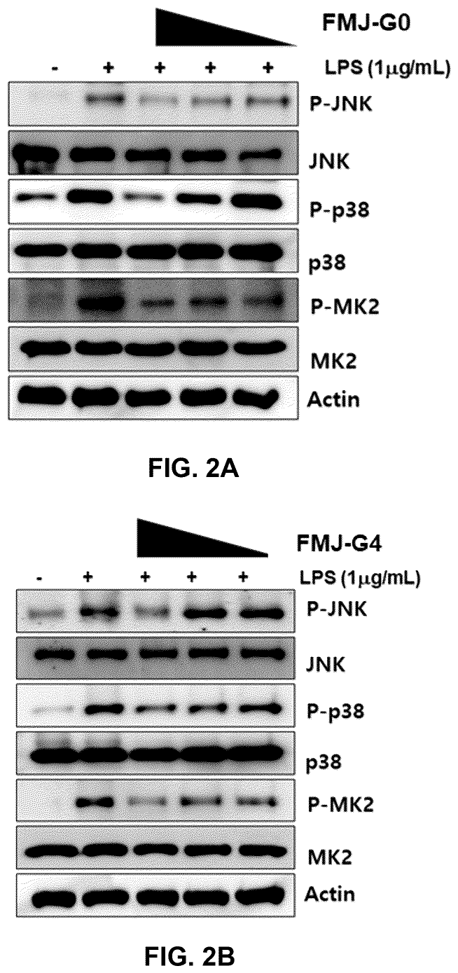

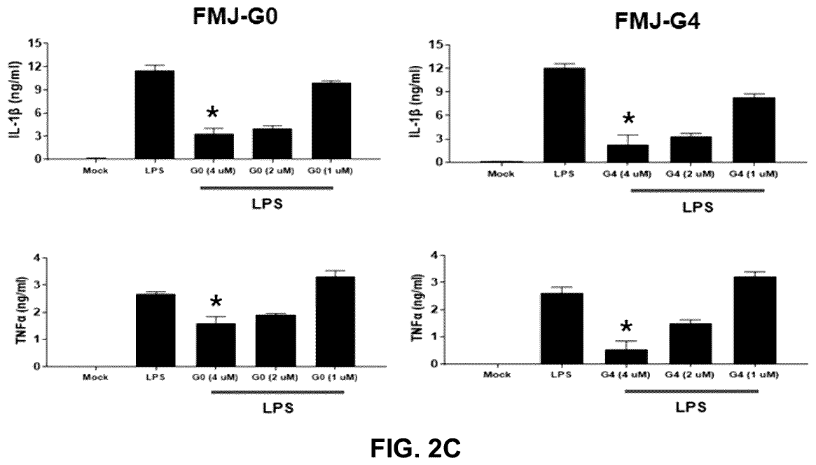

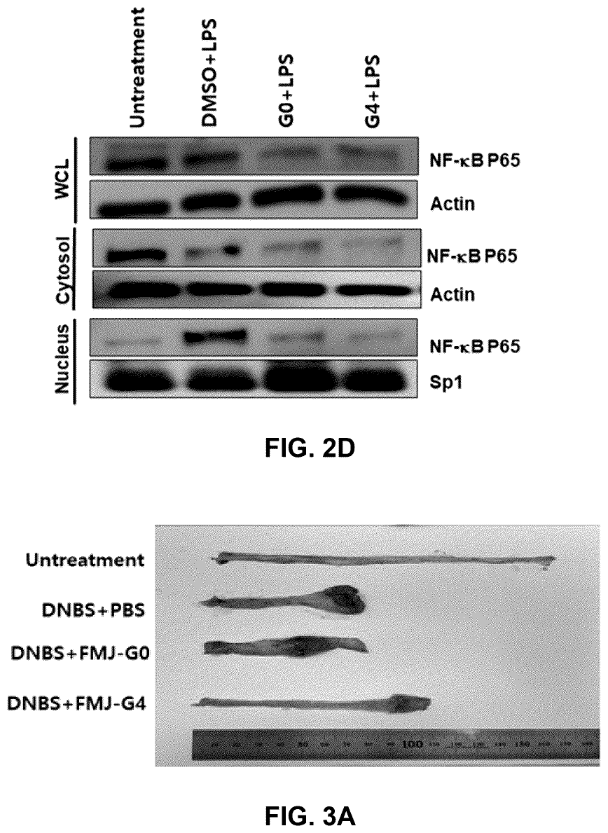

As a result, referring to FIGS. 2A and 2B, BMDA (FMJ-G0) and DMMA (FMJ-G4) as the derivative thereof as the low-molecular weight substances suppressed activities of the p38 MAPK and JNK activated by LPS, and inhibited the inflammation-related signaling pathway leading to p38 MAPK-MK2. Based on these results, it was identified that these low-molecular-weight compounds were concentration-dependent.

Further, the biological significance of the above result is as follows. It was identified based on ELISA (FIG. 2C) that BMDA (FMJ-G0) and a derivative thereof. DMMA (FMJ-G4) according to the present disclosure as a low molecular substance inhibited the production of TNF-α and IL-1β inflammatory cytokines. It was identified that BMDA (FMJ-G0) and a derivative thereof. DMMA (FMJ-G4) according to the present disclosure as a low molecular substance inhibited the activity of the transcription factor NF-_{K}B, and inhibited NF-_{K}B expression and migration in the nucleus (FIG. 2D).

2. Evaluation of anti-inflammatory efficacy of BMDA (FMJ-G0) and a derivative thereof (DMMA, FMJ-G4) in a DNBS-induced colitis model

For experimental colitis, male SD rats (250 to 260 g) were fasted one day before developing colitis. A water bottle was prepared so that only water could be consumed by the rats. Then, after anesthesia of the rats, a rubber cannula is inserted into the anus, and a solution in which 48.0 mg DNBS was dissolved in 0.4 mL of 50% alcohol was injected thereto through the cannula. This process was repeated for 3 days.

From the 4th day since the DNBS was inserted, anesthesia of the rats was carried out, and then BMDA (FMJ-G0) and a derivative thereof (DMMA, FMJ-G4) were diluted with PBS, and then 500 μL of the diluted substance was injected at a concentration of 200 μg/mL thereto through the anus. This process was repeated for 5 days. In a control group, DMSO was diluted in PBS at the same concentration and then was injected in the same way for 5 days. Thereafter, on day 6, the rats were suffocated, and the large intestine was removed therefrom to evaluate a level of colitis according to a known method.

Moreover, a portion of the large intestine was removed, placed in a cell lysis buffer (20 mM Tris-HCl [pH 7.5], 150 mM NaCl, 1 mM EDTA, 1% Triton X-100, 0.5% SDS), and subjected to ultrasonic treatment. Centrifugation was carried out to collect only the supernatant. We quantified the protein, and performed immunoblotting thereon.

Further, a portion of the supernatant obtained after the centrifugation as prepared for immunoblotting was used to measure the amount of the chemokine CINC-3 (Cytokine-induced neutrophil chemoattractant) based on ELISA. Moreover, in order to measure the level of activity of macrophages infiltrated into the distal colon, a titer of MPO (Myeloperoxidase) was measured. The distal colon was pulverized in a supersonic pulverizing device, was centrifuged to obtain the supernatant. Then, hydrogen peroxide was added to the supernatant for decomposition. Change in absorbance was measured at 460 nm for 1 minute.

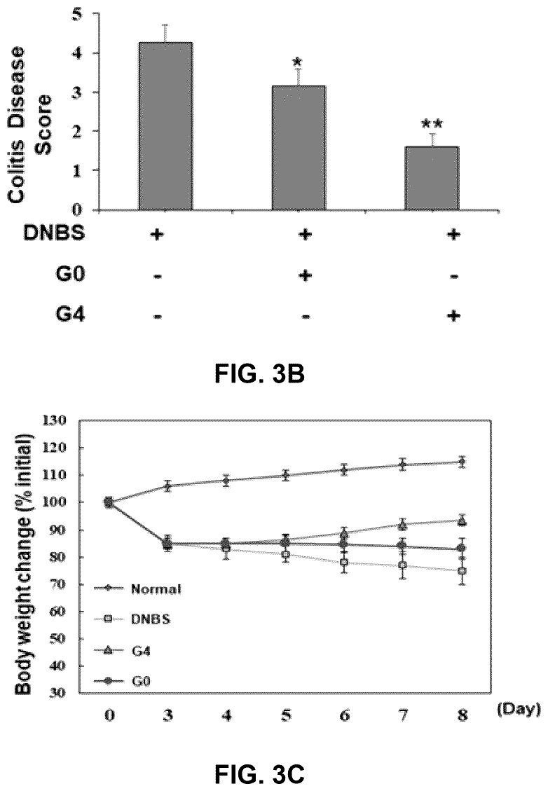

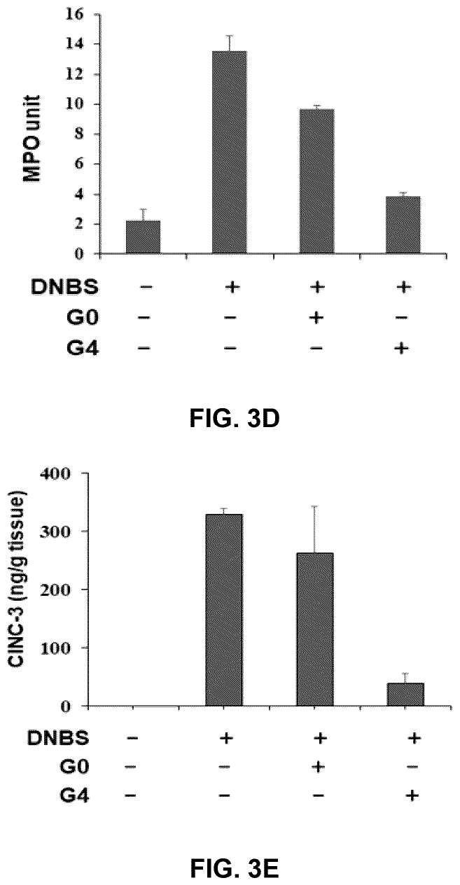

As a result, referring to FIGS. 3A to 3B, BMDA (FMJ-G0) and the derivative thereof. DMMA (FMJ-G4) as a low-molecular weight substance significantly reduced the inflammatory response caused by DNBS by a significant amount.

When the rat was treated with DNBS+PBS, the length of the large intestine thereof was very shortened, and the ulcer scab was considerably large (FIGS. 3A and 3B). However, it was identified that the length of the colon of DMMA (FMJ-G4)-treated rats was larger than that of the DNBS+PBS-treated rat group, and scab caused by the ulcer was reduced. In particular, the rats treated with the derivative DMMA (FMJ-G4) exhibited slightly larger colon length and smaller ulcer scab than those of the rats treated with BMDA (FMJ-G0). The BMDA (FMJ-G0)-treated rats exhibited no significant difference in the length of the large intestine, compared to the DNBS+PBS treated group, but exhibited the much smaller ulcer scab than that in the DNBS+PBS treated group. However, BMDA (FMJ-G0) and DMMA (FMJ-G4) treatments did not restore the body weight lost due to the DNBS treatment (FIG. 3C).

Further, the colon of the rat to which BMDA (FMJ-G0) and the derivative thereof. DMMA (FMJ-G4) as the low-molecular weight substance was administered in the enema administration manner exhibited macrophage peroxidase activity much lower than that of the colon of DNBS+PBS-treated rat (FIG. 3D). Further, in the rat to which BMDA (FMJ-G0) and the derivative thereof. DMMA (FMJ-G4) as the low-molecular weight substance was administered in the enema administration manner, the production amount of CNIC-3 as a chemokine that attracts neutrophils was low (FIG. 3E).

3. Anti-inflammatory effect of BMDA (FMJ-G0) and a derivative thereof (DMMA, FMJ-G4) in rheumatoid arthritis model induced by type 2 bovine collagen

Rheumatoid arthritis was induced in 7-week-old DBA/J1 male mice. We thoroughly mixed the type 2 collagen at a concentration of 2 mg/mL with CFA (Complete Freund's Adjuvant) in the mixing ratio 1:1. We injected 0.1 mL (100 μg) of the mixture intradermally to a position away 2 cm from a base of the tail. After 2 weeks, we mixed the type 2 collagen with IFA instead of CFA in the mixing ratio 1:1. We administered the mixture subcutaneously to the base of the tail in the same amount and volume as those in the first injection. After 3 days, we grouped the mice into 4 groups and performed second administration thereto.

In one example, BMDA (FMJ-G0) and a derivative thereof (DMMA, FMJ-G4) at a concentration of 2.5 mg/mL were orally administered to male mice in a volume of 200 μL (20 mg/body weight Kg) for 21 days. In a control group, soybean oil was orally administered thereto.

During the experiment, the insteps of the forelimbs and hindlimbs were measured with a caliper three times a week, and the mice were suffocated, and joint areas were prepared for immunohistochemical staining and immunoblotting. Moreover, the spleen was removed therefrom and a size thereof was measured. Further, blood was drawn through the hepatic portal vein to prepare serum. A specific method was as follows.
1) Paraffin sections were prepared as the joints including the ankles of the forelimbs. At this time, decalcification is performed first, followed by sectioning the same at a thickness of 4 μm, and H&E staining.
2) The cell lysis solution was added thereto, and then we crushed the tissue with a sonicator, which was prepared for immunoblotting.
3) Trisol solution was added to the joint tissue, the tissue was pulverized in a sonicator, and centrifuged, and the Trisol solution was used to precipitate proteins and DNA for entire RNA separation, and the entire RNA was collected from the supernatant obtained via the centrifugation. c-DNA was synthesized using oligo-dT primer and reverse transcriptase, and then, quantitative qRT-PCR and immunoblotting were performed thereon.

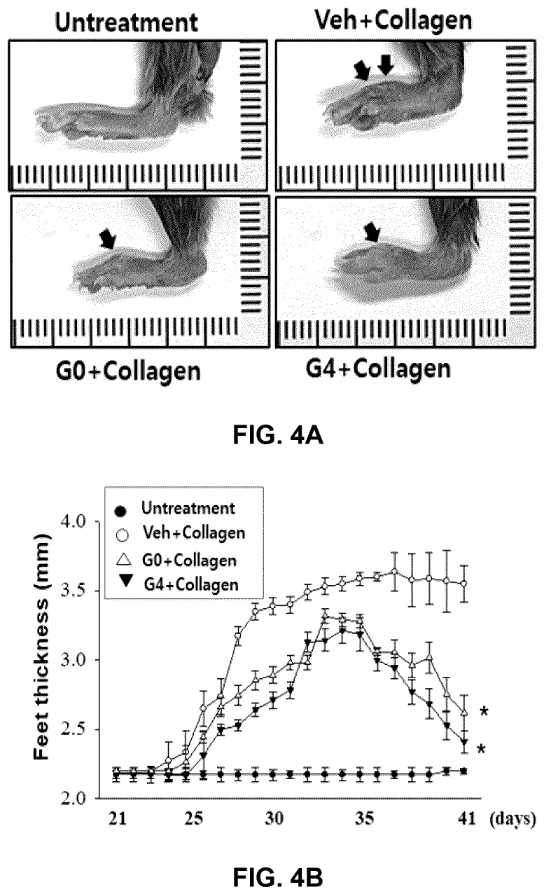

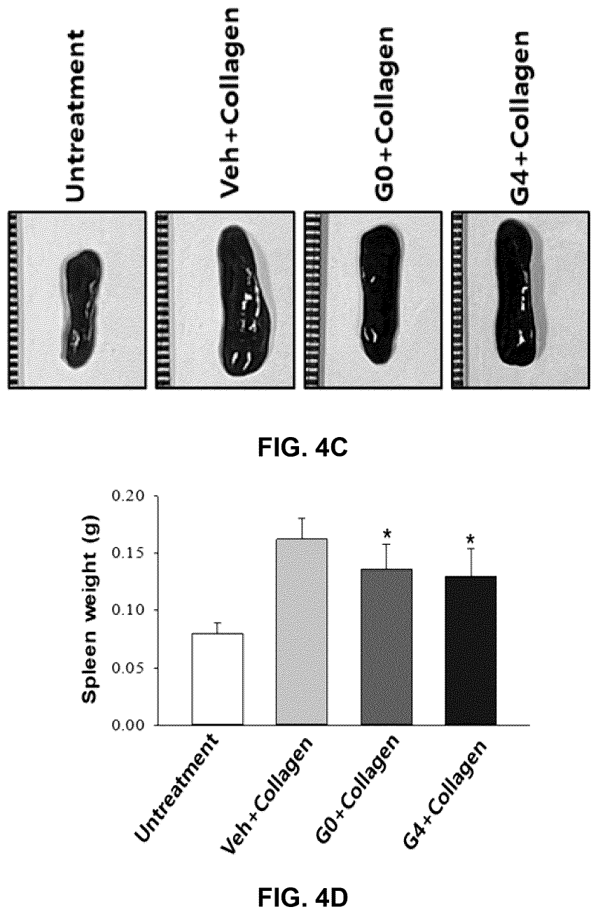

As a result, referring to FIG. 4A and FIG. 4B, the mice to which BMDA (FMJ-G0) and DMMA (FMJ-G4) were orally administered exhibited much smaller swelling of the feet, compared to the swelling of the feet of the control group using the soybean oil. Oral administration of BMDA (FMJ-G0) and DMMA (FMJ-G4) did not restore the weight loss caused by rheumatism induced by collagen (FIG. 4B). However, compared to the control group, the length and weight of the spleen of the rats treated with BMDA (FMJ-G0) and DMMA (FMJ-G4) were reduced (FIGS. 4C and 4D).

Further, referring to FIGS. 5A and 5B, the joint tissues were stained and examined. In this regard, severe inflammation and synovitis were observed in the rats having rheumatism induced by collagen to which the soybean oil was orally administered, whereas the oral administration of BMDA (FMJ-G0) and DMMA (FMJ-G4) significantly reduced the inflammation and synovitis. In particular, a significant reduction in inflammation was observed due to the oral administration of BMDA (FMJ-G0).

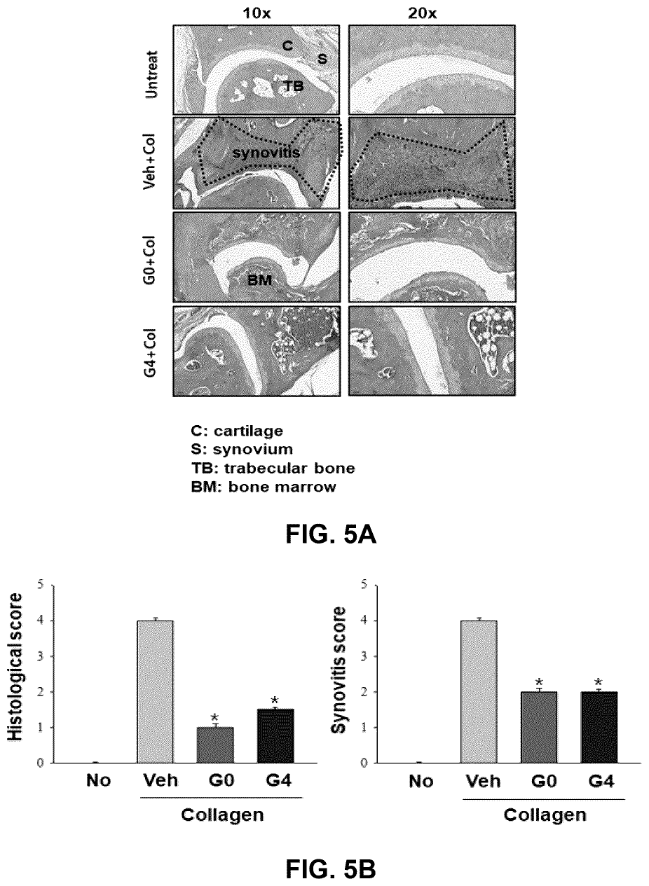

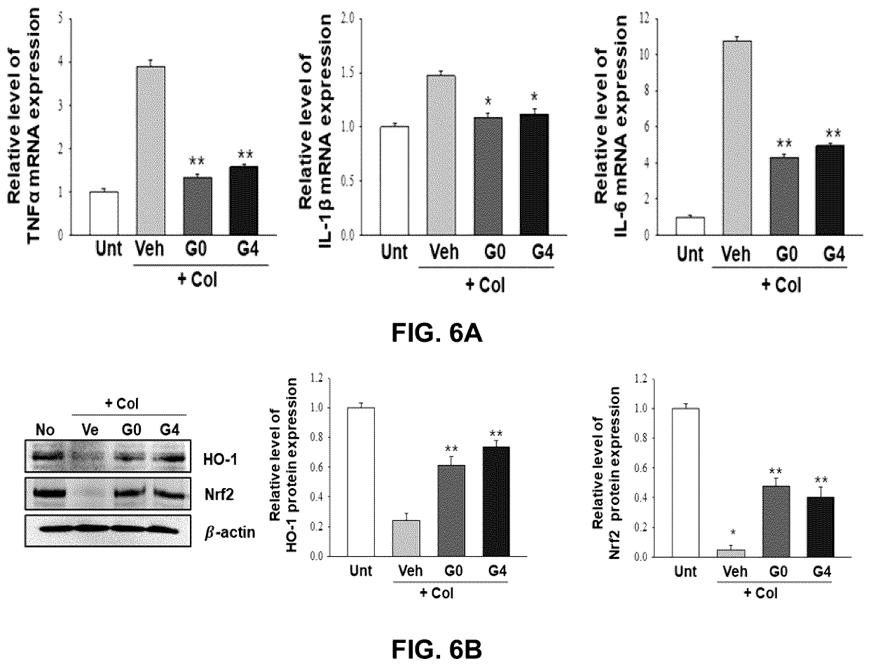

In one example, when the expression of inflammatory cytokines in the joint area was investigated based on qRT-PCR, low expression of inflammatory cytokines such as TNF-α, IL-1β, and IL-6 were observed in the spleen of mice to which the soybean oil was administered (FIG. 6A). Conversely, the expression of HO-1 and Nrf2 as two proteins involved in the antioxidant response was increased in the joints of animals to which BMDA (FMJ-G0) and DMMA (FMJ-G4) were orally administered (FIG. 6B).

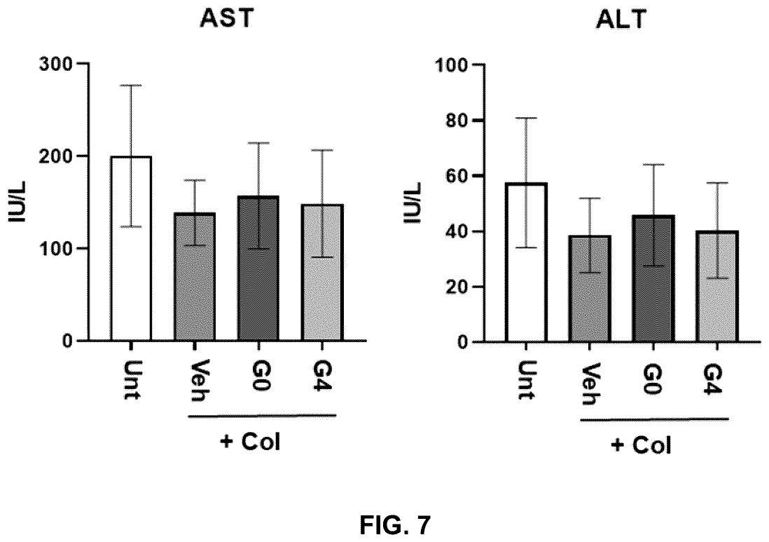

Above all, oral administration of BMDA (FMJ-G0) and DMMA (FMJ-G4) for 3 weeks did not increase the activity of AST and ALT enzymes known as indicators of hepatotoxicity. This 137608316.1-16 was identified based on the fact that the serum of the mouse subjected to the oral administration of BMDA (FMJ-G0) and DMMA (FMJ-G4) exhibited the activity of the AST and ALT enzymes similar to the activity of the AST and ALT enzymes which the serum of the mouse in the control group subjected to no treatment exhibited (see FIG. 7).

### <Example 3: Synthesis of Derivative of BMDA (FMJ-G0) and Evaluation of Anti-Inflammatory Efficacy Thereof

Benzyl-decyl-ethyl-amine (FMJ-G1), benzyl-dodecyl-methyl-amine (FMJ-G2), and (decyl-4-fluoro-benzyl)-methyl-amine (FMJ-G3) as the derivative of BMDA (FMJ-G0) were synthesized in the same manner as that of the Example 1 except for the reaction substance.

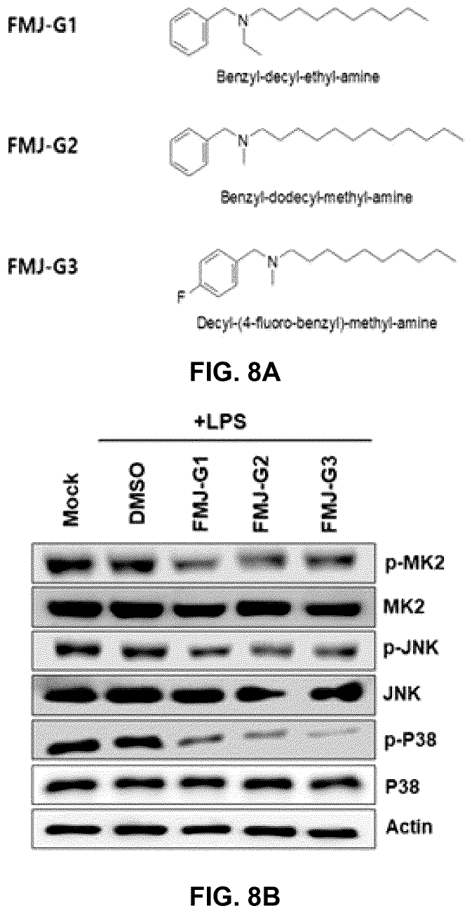

Specifically, the FMJ-G1 derivative was obtained by mixing the N-benzylethylamine and decanal at a concentration of 1M with each other in presence of methanol, and then performing reductive amination on the mixture and performing purification thereof through a column. The FMJ-G2 derivative was obtained by mixing N-benzylmethylamine and dodecanal at a concentration of 1M with each other in presence of methanol, and then performing reductive amination on the mixture and performing purification thereof through a column. In addition, the FMJ-G3 derivative was obtained by mixing 1-(4-fluorophenyl)-N-methylmethanamine and decanal at a concentration of 1M with each other in presence of methanol, and then performing reductive amination on the mixture and performing purification thereof through a column. The chemical structures of the synthesized substances benzyl-decyl-ethyl-amine (FMJ-G1), benzyl-dodecyl-methyl-amine (FMJ-G2), and (decyl-4-fluoro-benzyl)-methyl-amine (FMJ-G3) are respectively represented by following Chemical Formulas 1-3 to 1-5. (See FIG. 8A)

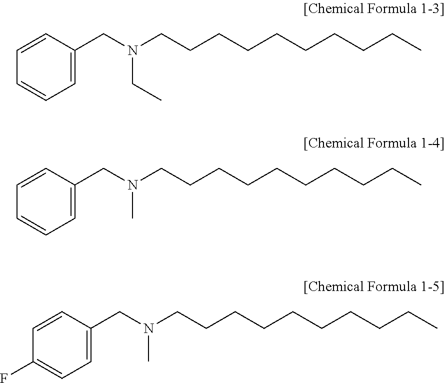 [SMILES: CCCCCCCCCCN(CC)Cc1ccccc1 ; CCCCCCCCCCN(C)Cc1ccccc1 ; CCCCCCCCCCN(C)Cc1ccc(F)cc1]

Thereafter, the THP-1 cell line was pretreated with the synthesized FMJ-G0 derivatives FMJ-G1, FMJ-G2, and FMJ-G3 at a concentration of 4 μM for 1 hour. Lipopolysaccharide (1 μg/mL) was added to a culture medium in which the THP-1 cell line was cultured for 4 hours. The same cell line was collected, and a signaling pathway involved in inflammatory cytokine synthesis was identified, and the result is shown in FIG. 8B.

As a result, it was identified that the pretreatment with FMJ-G1, FMJ-G2, and FMJ-G3 inhibited phosphorylation of p38 MAP kinase, JNK, and MK2 resulting from lipopolysaccharide (LPS), and the FMJ-G0 derivative compound suppressed the inflammatory response.

### <Example 4: Preparation of Cosmetic Composition Containing BMDA (FMJ-G0) and a Derivative Thereof and Evaluation of Atopic Dermatitis Ameliorating Efficacy Thereof

1. Cosmetic Composition Preparation

Wed added 0.3 g of olive wax to 2 mL of soybean oil (Sigma-Aldrich) in a glass tube (15 ml), and put the tube in a 75° C. water-bath, and performed a double boiling thereon to melt the olive wax for 30 minutes while vortexing the tube at 10 minute interval. Under the same conditions, 7.7 ml of purified water was put into a 75° C. water-bath and heated the same for 30 minutes. Next, 7.5 ml of purified water was poured into the tube containing the olive wax and the mixture was stirred well via vortexing. Then, 30 μg (0.3%) of each of BMDA and derivatives thereof (FMJ-G0, FMJ-G4, FMJ-G1, FMJ-G2, FMJ-G3) as synthesized in Examples 1 and 3 were added to the mixture, followed by stirring. The reaction product was cooled in ice and prepared in the form of a cosmetic cream.

2. Evaluation of Atopic Dermatitis Ameliorating Efficacy

In order to induce atopic dermatitis using 5-week-old Balb/C mice, hair on the back of the mouse was removed therefrom, and after one day, a 0.5% solution in which DNCB (2,4-dinitrochlorobenzne) is mixed with acetone and olive oil (3:1) was prepared, and then was applied thereon for 3 consecutive days by 150 μL per each day. Thereafter, the solution of the same concentration was applied to the back area from which the hair was removed twice a week and this process continued for 2 weeks. Further, to alleviate atopic dermatitis induced in this way, the cosmetic cream containing each of BMDA (FMJ-G0) and the derivatives thereof (FMJ-G4, FMJ-G1, FMJ-G2, FMJ-G3) of the present disclosure as prepared above was applied to the back of rats with the induced atopic dermatitis for 2 weeks, and then, changes in skin inflammation were examined.

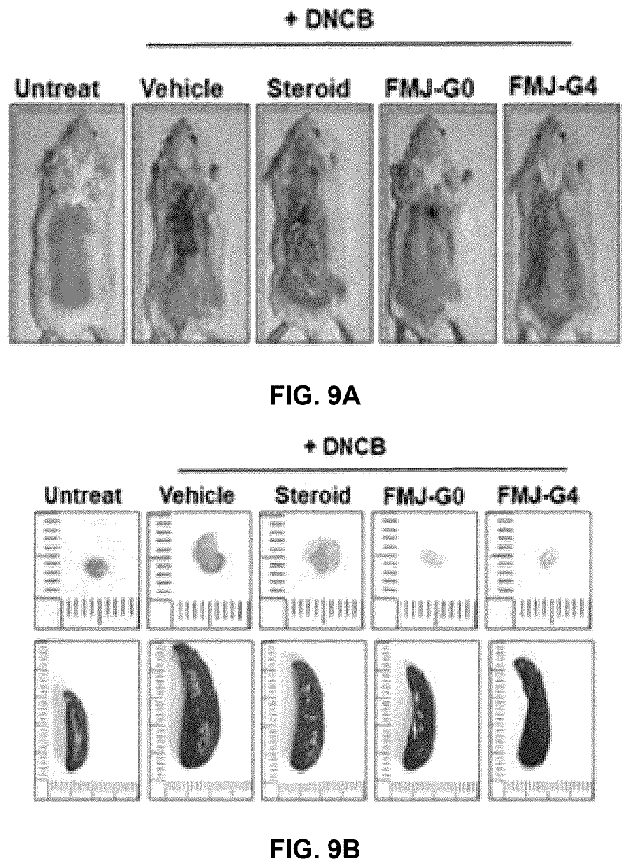

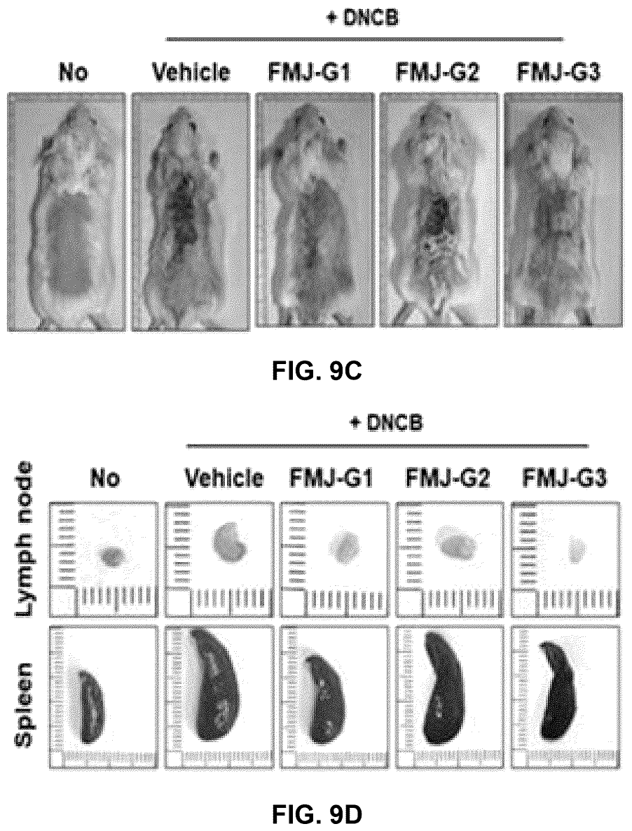

As a result, as shown in FIG. 9A, the cosmetic cream containing each of BMDA (FMJ-G0) and DMMA (FMJ-G4) significantly alleviated the DNCB-induced atopic dermatitis, and hair regrowth on the atopic dermatitis alleviated skin was observed. Moreover, in FIG. 9B, it was observed that immune cells were activated in the acute atopic dermatitis induced by DNCB and thus the spleen was larger. It was observed that a size of the spleen of the mouse of the skin onto which the cosmetic cream containing each of BMDA (FMJ-G0) and DMMA (FMJ-G4) was applied was similar to that of the mouse in which the atopic dermatitis was not induced. An experiment result of a size of the inguinal lymph node was the same as that of the spleen.

In one example, it was identified that when a commercial steroid ointment (Lidomax) (0.15%) was applied to the skin under the same conditions, the level of dermatitis, the size of the spleen, and the size of the lymph were similar to those of the vehicle of the cosmetic cream that was free of the active ingredient. (FIG. 9A and FIG. 9B).

Moreover, referring to FIG. 9C showing the results of applying the cosmetic cream containing the derivative synthesized in Example 3, it may be observed that the application of each of FMJ-G1 and FMJ-G3-containing cosmetic creams significantly alleviates atopic dermatitis. However, the treatment with the FMJ-G2-containing cosmetic cream did not prevent the progression of acute dermatitis caused by DNCB as in the control group (Vehicle). These results have a very close relationship with the sizes of the spleen and the lymph. That is, referring to FIG. 9D, it may be identified that the size of each of the spleen and the lymph of the mouse to which the FMJ-G2 containing cosmetic cream is applied has been increased as the size of each of the spleen and the lymph of the mouse treated with the control group (Vehicle) has been increased.

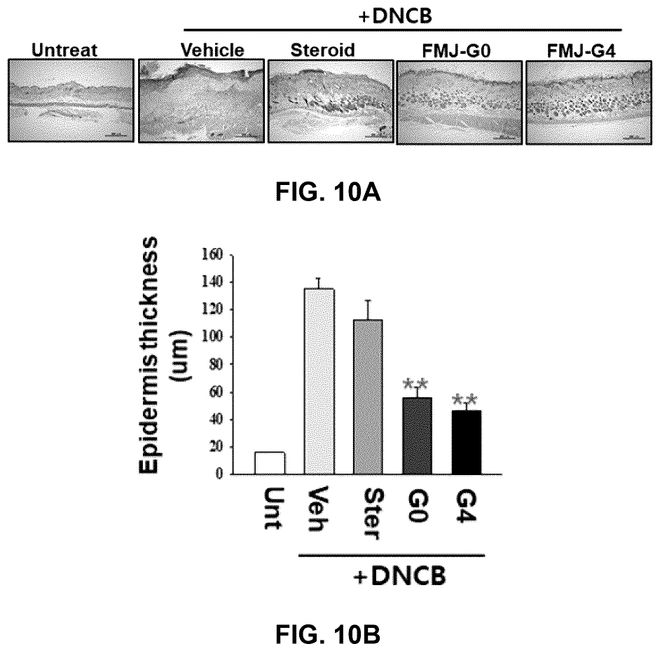

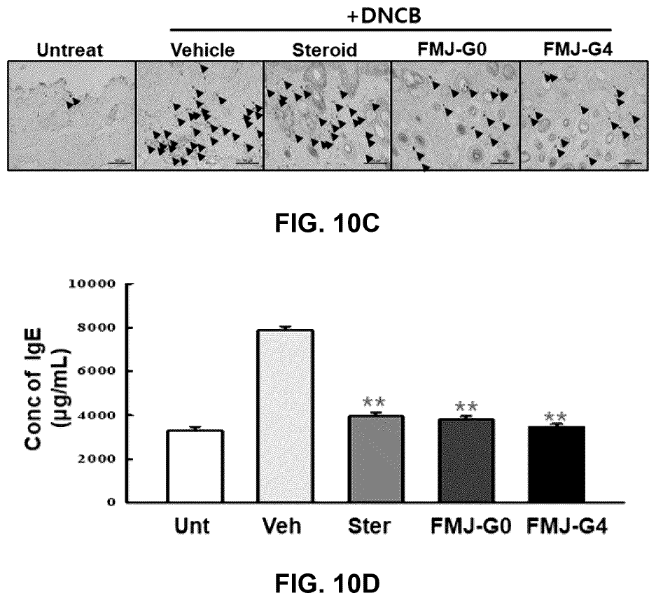

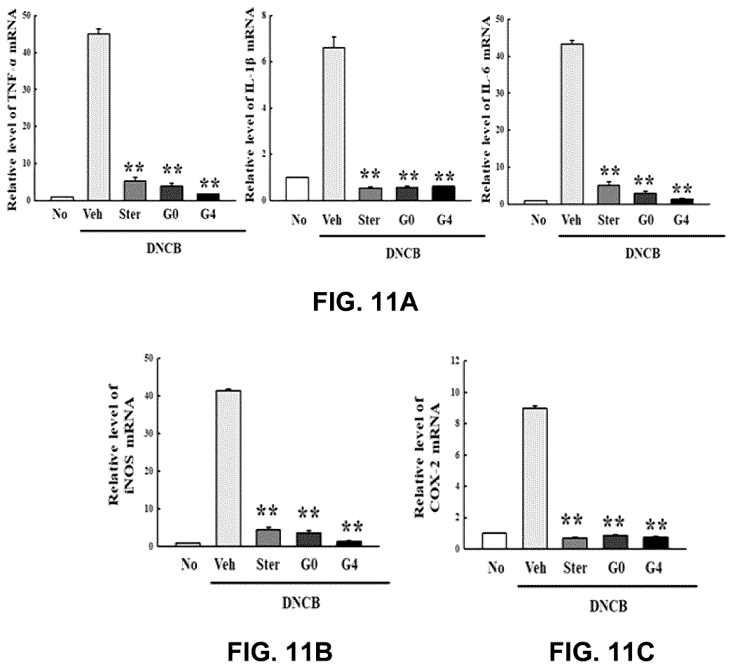

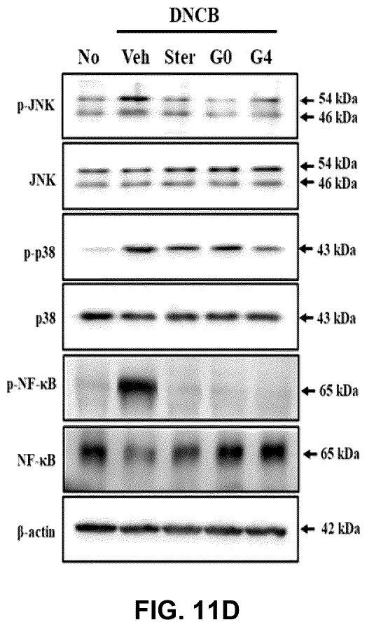

FIGS. 10A and 10B show that when each of BMDA (FMJ-G0) and DMMA (FMJ-G4) is applied to the skin, acute dermatitis caused by DNCB is significantly prevented such that a thickness of the dermis is significantly reduced.

The experimental results of FIG. 10A and FIG. 10B was obtained as follows as in FIG. 9. The skin tissue was removed from the back of the mouse in the group, and was placed fixedly in 10% formaldehyde for 48 hours, and paraffin pieces were made, and the pieces were thinly sliced so as to have a thickness of 4 μm and was subjected to hematoxylin-esoin staining. Then, whether the damaged skin tissue was recovered by each of BMDA (FMJ-G0) and DMMA (FMJ-G4) was examined using an optical microscope equipped with a Leica application (Leica Microsystems, Switzerland).

It is identified that the treatment with BMDA or DMMA (FMJ-G0/G4) induces a decrease in the thickness of the dermal layer due to inflammation caused by DNCB. The dermal layer is the second layer from the top in the photo in FIG. 10A. The first dark purple layer is the epidermis layer, the light pink layer thereunder is the dermis layer, and the dark color thereunder is the subcutaneous layer, and a portion thereunder is the muscle.

Referring to FIG. 10C, it is identified via toluidine-blue staining that the treatment with each of BMDA (FMJ-G0) and DMMA (FMJ-G4) prevents invasion of mast cells into the dermis, thereby suppressing the inflammatory response in the dermis. FIG. 10C shows the result of observing, under an optical microscope, the mast cells that have invaded the dermal layer via staining a portion of the thin slices prepared in FIG. 10A-B with toluidine-blue solution. Each of BMDA (FMJ-G0) and DMMA (FMJ-G4) prevents the invasion of mast cells into the dermal layer to prevent inflammation in the dermal layer from mast cells.

FIG. 10D shows that the concentration of IgE which is closely related to allergic reactions in the blood of mice treated with each of BMDA or DMMA (FMJ-G0/G4) is significantly lower compared to that of the control group. To this end, the atopic dermatitis is induced in FIG. 9, and on 2 weeks since the treatment with BMDA (FMJ-G0) and DMMA (FMJ-G4), blood (500 ul) is collected from the hepatic veins of the rats of the above groups, and left at room temperature for 30 minutes. Then, serum was separated and prepared via centrifugation, and then, the IgE concentration related to the allergic reaction was measured through ELISA using the serum thus prepared.

Anti-mouse IgE (Pharmingen, CA, USA) was diluted with PBS (pH 7.4) on a 96-well plate, 100 μL thereof was coated on each 96-well plate, and then left at 4° C. for 12 hours. The plate was washed with PBS containing 0.05% tween. Blocking was performed thereon for 1 hour with PBS containing 1% BSA, 5% sucrose, and 0.05% NaN₃. Washing was performed thereon several times using washing buffer. Recombinant IgE and serum were added thereto, and reaction occurred at 37° C. for 2 hours, and each well was washed again, and biotinylated IgE (Pharmingen, CA, USA) was added thereto. Reaction occurred for 2 hours. After washing the well, avidin peroxidase was added thereto and reaction occurred at 37° C. for 30 minutes. Then, the well was washed and then, ABTS solution as a substrate was added thereto. Absorbance was measured at 405 nm using ELISA reader (Epoch2, Biotek, VT, USA). It may be identified that the treatment with BMDA or DMMA (FMJ-G0/G4) also inhibits IgE production related to allergic reactions.

FIG. 11 show data obtained as follows. The atopic dermatitis was induced as described in FIG. 9, and on 2 weeks since the treatment with BMDA or DMMA (FMJ-G0/G4), a portion of the skin tissue of the rats in the groups were put in Trisol solution and the tissue was crushed in a sonicator. Centrifugation was performed thereon, and protein and DNA are precipitated in Trisol solution for entire RNA isolation, and centrifugation was performed thereon to obtain the supernatant from which entire RNA was collected. c-DNA was synthesized therefrom using oligo-dT primer and reverse transcriptase. Quantitative data about amounts of transcriptomes TNF-α, qRT-PCR for IL-1β, IL-6, iNOS, and Cox2 was measured based on qRT-PCR.

In the skin of the control group treated with DNCB, the amounts of transcriptomes of the proteins iNOS and COX2 that produce inflammatory cytokines TNF-α, IL-1β, and IL-6, and inflammatory mediators (NO, PDE2) were rapidly increased. In this regard, treatment with BMDA or DMMA (FMJ-G0/G4) markedly lowered the amount of the transcriptomes thereof (FIG. 11A-C).

When the activity of signaling proteins that affect the transcription of these proteins was investigated using immunoblotting, BMDA or DMMA (FMJ-G0/G4) treatments significantly lowered the phosphorylation level of JNK, p38MAPK and NF-_{K}B which are related to inflammation, as supported by the previous results, compared to the control group (FIG. 11D).

Although the embodiments of the present disclosure have been described in more detail with reference to the accompanying drawings, the present disclosure is not necessarily limited to these embodiments, and may be modified in a various manner within the scope of the technical spirit of the present disclosure. Accordingly, the embodiments as disclosed in the present disclosure are intended to describe rather than limit the technical idea of the present disclosure, and the scope of the technical idea of the present disclosure is not limited by these embodiments. Therefore, it should be understood that the embodiments described above are not restrictive but illustrative in all respects.

What is claimed is:

1. An anti-inflammatory composition containing, as an active ingredient, a compound represented by a following Chemical Formula 1: 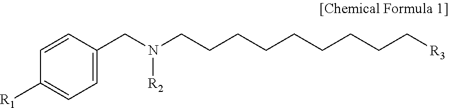
wherein in the Chemical Formula 1,
R₁ represents hydrogen, fluorine, an alkyl group having 1 to 5 carbon atoms, or an alkoxy group having 1 to 5 carbon atoms,
each of R₂ and R₃ independently represents an alkyl group having 1 to 5 carbon atoms.

2. The anti-inflammatory composition of claim 1, wherein the anti-inflammatory composition contains one selected from compounds respectively represented by following Chemical Formulas 1-1 to 1-5 as the active ingredient:

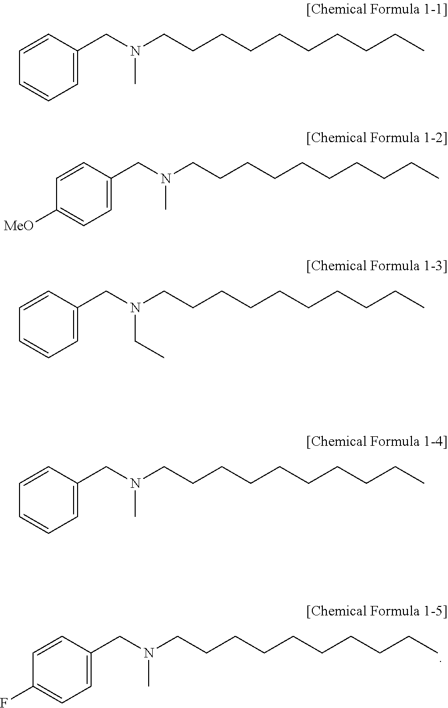 [SMILES: CCCCCCCCCCN(C)Cc1ccccc1 ; CCCCCCCCCCN(C)Cc1ccc(OC)cc1 ; CCCCCCCCCCN(CC)Cc1ccccc1 ; CCCCCCCCCCN(C)Cc1ccccc1 ; CCCCCCCCCCN(C)Cc1ccc(F)cc1]

3. The anti-inflammatory composition of claim 1, wherein the anti-inflammatory composition has a therapeutic effect on an inflammatory disease including atopic dermatitis, rheumatoid arthritis, or ulcerative colitis.

4. The anti-inflammatory composition of claim 1, wherein the anti-inflammatory composition inhibits activation, expression and production of p38 MAP kinase and JNK induced by lipopolysaccharide, and inhibits an inflammation-related signal transduction pathway linked to p38 MAPK-MK2.

5. The anti-inflammatory composition of claim 1, wherein the anti-inflammatory composition inhibits production of a pro-inflammatory cytokine selected from TNF-α, IL-1β or IL-6, and inhibits activity of a transcription factor NF-_{K}B.

6. The anti-inflammatory composition of claim 1, wherein the anti-inflammatory composition increases expression of HO-1 (Heme oxygenase 1) or Nrf2 (nuclear factor erythroid-related factor 2), thereby achieving an antioxidant effect.

7. The anti-inflammatory composition of claim 1, wherein the anti-inflammatory composition inhibits production of chemokine CINC-3.

8. A cosmetic composition for ameliorating atopic dermatitis, the composition containing, as an active ingredient, a compound represented by a following Chemical Formula 2: 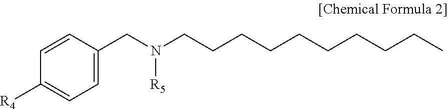
wherein in the Chemical Formula 2,
R₄ represents hydrogen, fluorine, an alkyl group having 1 to 5 carbon atoms, or an alkoxy group having 1 to 5 carbon atoms,
R₅ represents an alkyl group having 1 to 5 carbon atoms.

9. The cosmetic composition of claim 8, wherein the cosmetic composition contains one selected from compounds respectively represented by following Chemical Formulas 1-1, 1-2, 1-3 and 1-5 as the active ingredient:

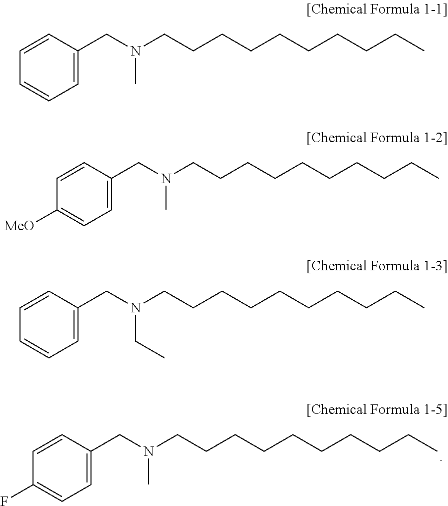 [SMILES: CCCCCCCCCCN(C)Cc1ccccc1 ; CCCCCCCCCCN(C)Cc1ccc(OC)cc1 ; CCCCCCCCCCN(CC)Cc1ccccc1 ; CCCCCCCCCCN(C)Cc1ccc(F)cc1]

10. The cosmetic composition of claim 8, wherein the cosmetic composition further contains at least one selected from a group consisting of a skin moisturizing ingredient, a cosmetic formulation ingredient, a fragrance, a preservative, and a purified water.

11. The cosmetic composition of claim 8, wherein a formulation of the cosmetic composition is lotion, cream, emulsion, essence, gel, serum, pack, powder, skin ointment, skin patch, suspension, spray, or cosmetic solution.

12. A pharmaceutical composition for preventing or treating rheumatoid arthritis, the composition containing, as an active ingredient, a compound represented by a following Chemical Formula 1 or a pharmaceutically acceptable salt thereof: 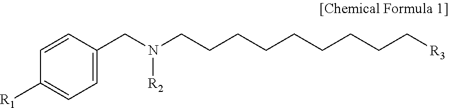
wherein in the Chemical Formula 1,
R₁ represents hydrogen, fluorine, an alkyl group having 1 to 5 carbon atoms, or an alkoxy group having 1 to 5 carbon atoms,
each of R₂ and R₃ independently represents an alkyl group having 1 to 5 carbon atoms.

13. The pharmaceutical composition of claim 12, wherein the pharmaceutical composition contains a compound represented by a following Chemical Formula 1-1 or Chemical Formula 1-2 as the active ingredient:

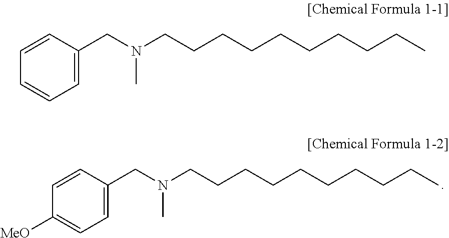 [SMILES: CCCCCCCCCCN(C)Cc1ccccc1 ; CCCCCCCCCCN(C)Cc1ccc(OC)cc1]

14. The pharmaceutical composition of claim 13, wherein the compound represented by Chemical Formula 1 is the compound represented by the Chemical Formula 1-1.

15. The pharmaceutical composition of claim 12, wherein the pharmaceutical composition is administered orally.

16. The pharmaceutical composition of claim 15, wherein the compound represented by the Chemical Formula 1 is formulated in a unit dosage form suitable for oral administration at a dose of 15 to 25 mg/kg.

17. A medicinal composition for treatment of ulcerative colitis, the composition containing, as an active ingredient, a compound represented by a following Chemical Formula 1 or a pharmaceutically acceptable salt thereof: 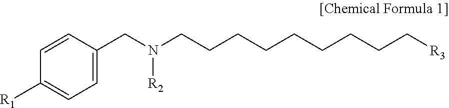
wherein in the Chemical Formula 1,
R₁ represents hydrogen, fluorine, an alkyl group having 1 to 5 carbon atoms, or an alkoxy group having 1 to 5 carbon atoms,
each of R₂ and R₃ independently represents an alkyl group having 1 to 5 carbon atoms.

18. The medicinal composition of claim 17, wherein the medicinal composition contains, as the active ingredient, a compound represented by a following Chemical Formula 1-1 or Chemical Formula 1-2:

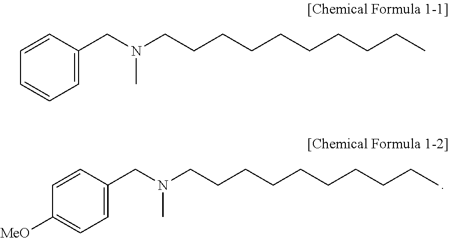 [SMILES: CCCCCCCCCCN(C)Cc1ccccc1 ; CCCCCCCCCCN(C)Cc1ccc(OC)cc1]

19. The medicinal composition of claim 18, wherein the compound represented by the Chemical Formula 1 is the compound represented by the Chemical Formula 1-2.

20. The medicinal composition of claim 17, wherein the medicinal composition is administered in an enema manner.

21. The medicinal composition of claim 20, wherein the medicinal composition is administered directly to an ulcerative colitis patient in the enema manner at 0.4 mg/kg or smaller as an effective dosage per day.

22. The medicinal composition of claim 17, wherein administration of the medicinal composition in an enema manner suppresses production of chemokine CINC-3.
